(19) United States
(12) Patent Application Publication
(10) Pub. No.: US 2003/0157154 A1
(43) Pub. Date: Aug. 21, 2003

(54) Compositions containing hydroxy aromatic aldehydes and their use in treatments

(76) Inventors: Bryan Fuller, Edmond, OK (US); Brian Keith Pilcher, Edmond, OK (US)

Correspondence Address: William H. Benz, Esq., BURNS, DOANE, SWECKER & MATHIS, L.L.P., P.O. Box 1404, Alexandria, VA, 22313-1404, US

(21) Appl. No.: 10/222,207

(22) Filed: Aug. 15, 2002

Related U.S. Application Data
(60) Provisional application No. 60/346,049, filed on Jan. 4, 2002.
(60) Provisional application No. 60/368,565, filed on Apr. 1, 2002.
(60) Provisional application No. 60/384,690, filed on May 30, 2002.

(51) Int. Cl. A61K 9/70; A61K 31/11

(52) U.S. Cl. 424/449; 514/699

(57) ABSTRACT

Disclosed are pharmaceutical and cosmetic compositions containing hydroxy aromatic aldehyde compounds. The disclosed compositions are useful as topical therapeutics for treating inflammatory dermatologic conditions. The disclosed compositions are also useful in transdermal and other systemic dose forms for treating other inflammatory conditions in mammals.

### CROSS-REFERENCE TO RELATED APPLICATIONS

This application claims priority under 35 U.S.C. 119(e) to U.S. Provisional application Serial No. 60/346,049 (Attorney Docket Number 032277-026) which was filed on Jan. 4, 2002, and to U.S. Provisional Application Serial No. 60/368,565 (Attorney Docket Number 032277-030) which was filed on Apr. 1, 2002, and to U.S. Provisional Application Serial No. 60/384,690 (Attorney Docket Number 032277-033) which was filed on May 30, 2002, the disclosures of which are incorporated herein in their entirety.

### BACKGROUND OF THE INVENTION

1. Field of the Invention

This invention relates to aromatic aldehydes and their use as active ingredients in cosmetics and pharmaceuticals. More particularly it concerns such aldehydes and their use in cosmetics and as topical, transdermal or systemic pharmaceuticals.

2. State of the Art

This invention involves the use of aromatic aldehydes. Many aromatic aldehydes are known materials that commonly find use as chemical intermediates. Some aromatic aldehydes are components of natural products as well.

The present invention uses these aldehydes as active ingredients in pharmaceuticals and cosmetics. While the invention contemplates that these aldehyde materials can find application as systemic agents against inflammatory conditions when delivered transdermally or orally or by injection, at this time their preferred uses are as components of topical cosmetic and pharmaceutical compositions used to treat a wide range of dermatological conditions ranging from dermatitis and U.V.—induced inflammation through psoriasis and acne.

Therapies used in the past to deal with conditions such as eczema and psoriasis have included the use of simple emollients. Topical steroids ranging from mild agents such as hydrocortisone (1%) through more potent materials such as clobetasol propionate (0.05%) have been indicated with the common inflammatory dermatoses. In addition, corticosteroids and immunosuppresents have been used to treat skin conditions. Vitamin D and its derivatives such as calcipotrial and tacolcitol and vitamin A and other retinoids have been used to treat dermatological problems. The vitamin D materials are used to treat acne.

In addition to those directly topical therapies, it is well known that many materials pass through the skin and enter the systemic circulation when placed on the skin. The line between “topical” and this so called “transdermal” administration of drugs is a fuzzy one and many therapies heretofore have had both topical and transdermal aspects.

These therapies are not without their limitations. Emollients are very temporary and must be repeatedly renewed. Topical steroid use is associated with thinning skin, bruising, and rashes as well as serious systemic side effects such as development of Cushing's Syndrome.

The vitamin D materials often pass transdermally and can have unexpected effects on the user's systemic calcium metabolism. The retinoids are reported to cause acne in some cases and to produce teratogenic effects if absorbed transdermally during pregnancy.

It is clear that there is a need for additional topical compositions which can effectively treat dermatological conditions. It would be highly desirable if these compositions could also treat optionally transdermally or otherwise systemically treatable inflammatory conditions and avoid some or all of the problems associated with therapies now in use.

### SUMMARY OF THE INVENTION

It has now been found that a group of aromatic aldehydes are effective topical agents against inflammation-related dermatological conditions. These aldehydes also appear to be delivered to a measurable extent transdermally and thus to potentially achieve systemic and/or localized anti-inflammatory effects within the body. In view of these findings, it further appears that these aldehydes can be effective against other inflammatory conditions when administered by other systemic routes, as well.

In one of its composition aspects, this invention is directed to topical pharmaceutical and cosmetic compositions containing a pharmaceutically-acceptable topical carrier and one or more aromatic aldehyde compounds. These aromatic aldehydes include materials of Formula I, as well as protected versions, that is, acetals as in Formula II, and hemiacetals as in Formula III:

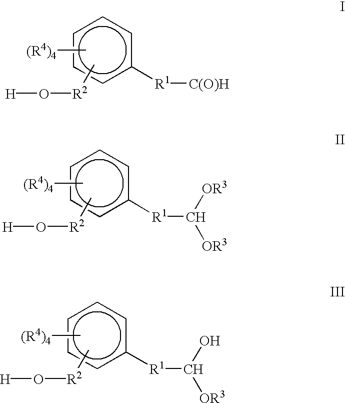

wherein

R¹ is a carbon-carbon single bond or a straight chain or branched chain alkylene;

R² is a carbon-oxygen single bond, or a straight chain or branched chain alkylene;

each R³ is independently alkyl, or in the case of the acetals of Formula II, the two R³s together with the atoms to which they are attached form a heterocycloalkyl; and

each R⁴ is independently selected from the group consisting of hydrogen, hydroxyl, alkyl, substituted alkyl, alkcycloalkyl, cycloalkyl, alkoxy, alkcycloalkoxy, cycloalkoxy, acyl, acyloxy and halogen.

In another of its composition aspects, this invention is directed to pharmaceutical compositions for topical, transdermal or other systemic administration containing a pharmaceutically-acceptable carrier and one or more of the aromatic aldehyde compounds of Formula I, II or III.

In one of its method aspects, this invention is directed to a method for treating a patient with a dermatological disease which method comprises topically administering to said patient a pharmaceutical composition comprising a pharmaceutically acceptable topical carrier and an effective dermatological disease-treating amount of a compound of Formula I, II or III above.

In another one of its method aspects, this invention is directed to a method for treating a dermatological condition, which method comprises topically applying to a human a cosmetic composition comprising a pharmaceutically acceptable topical carrier and an effective amount of a compound of Formula I, II or III above.

In still another of its method aspects, this invention is directed to a method for treating a patient with an inflammatory disease which method comprises systemically administering to said patient a pharmaceutical composition comprising a pharmaceutically acceptable carrier and an effective inflammatory disease-treating amount of a compound of Formula I, II or III above.

In yet another of its method aspects, this invention is directed to a method for treating a human with an inflammatory condition which method comprises topically applying to said human a pharmaceutical composition comprising a pharmaceutically acceptable carrier and an effective amount of a compound of Formula I, II or III above.

In yet another of its method aspects, this invention is directed to a method for improving the skin appearance of a patient which method comprises topically administering to said patient a pharmaceutical composition comprising a pharmaceutically acceptable carrier and a pharmaceutically effective amount of a compound of Formula I, II or III above.

In another one of its method aspects, this invention is directed to a method for improving the skin appearance of a human which method comprises topically applying to said human a pharmaceutical composition comprising a pharmaceutically acceptable carrier and an effective amount of a compound of Formula I, II or III above.

### DETAILED DESCRIPTION OF THE INVENTION

Brief Description of the Drawing

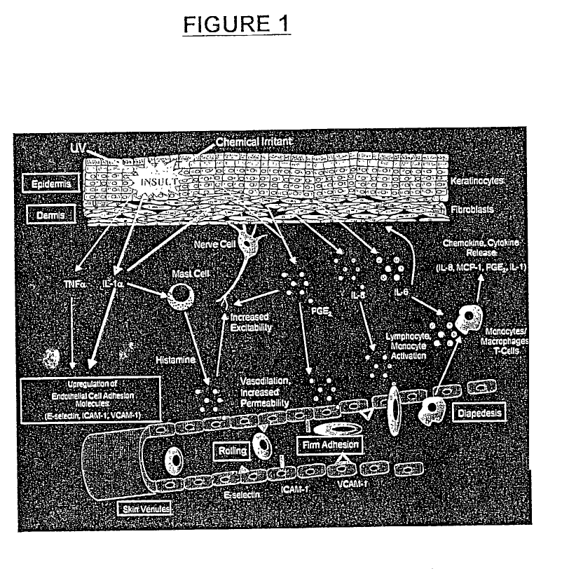

FIG. 1: A schematic diagram illustrating inflammatory processes in the skin and showing the relationship of inflammation to the release of various proteins.

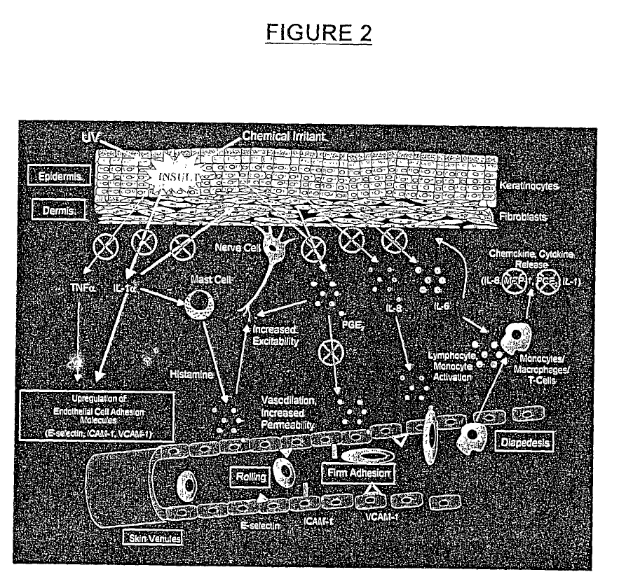

FIG. 2: A repeat of FIG. 1 illustrating those inflammatory processes which are effectively treated using the present invention.

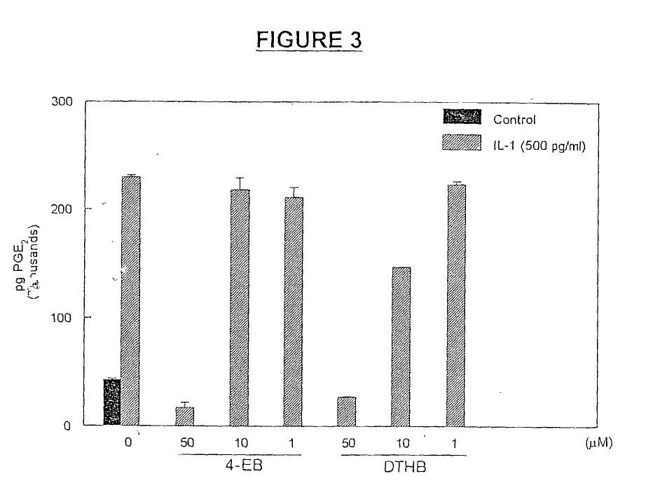

FIG. 3: A bar graph that shows the effects of 3,5 di-tert-butyl-4-hydroxybenzaldehyde, (“DTHB”) and 4-ethoxybenzaldehyde (4-EB) on interleukin 1, (“IL-1”) induced prostaglandin E2 “PGE₂” in fibroblasts.

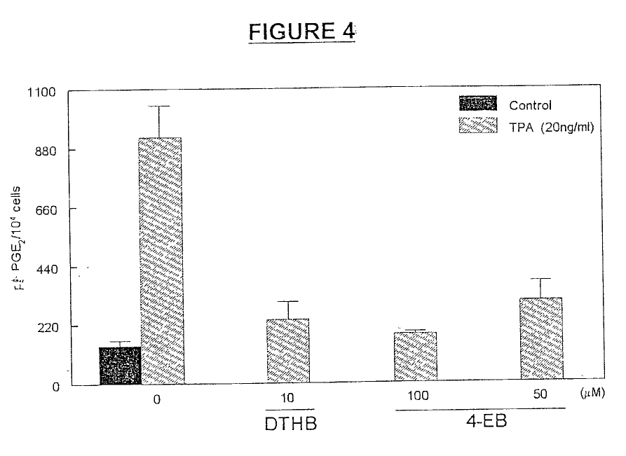

FIG. 4: A bar graph that shows the effects of DTHB and 4-EB on tetradecanoyl phorbol acetate (“TPA”)-induced PGE₂ in keratinocytes.

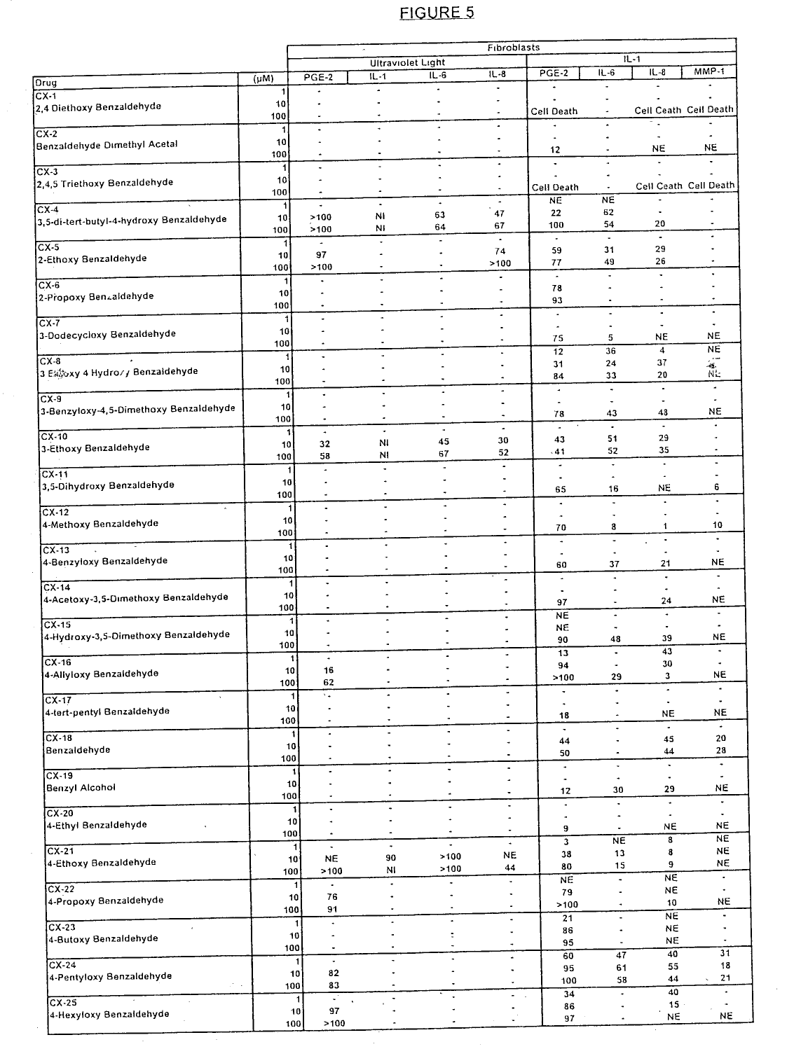

FIG. 5: A table which shows the effects of aldehydes employed in the compositions of this invention and other related compounds on expression levels of varius proteins in fibroblasts challenged with IL-1 or UV light.

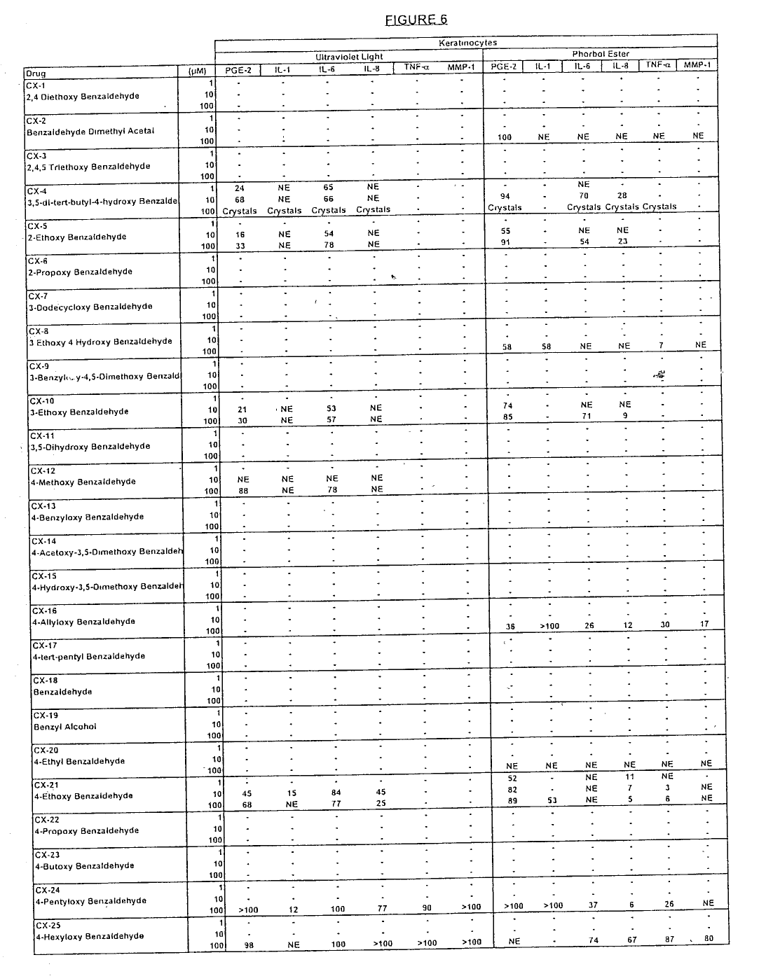

FIG. 6: A table which shows the effects of aldehydes employed in the compositions of this invention and other related compounds on expression levels of varius proteins in keratinocytes challenged with TPA or UV light.

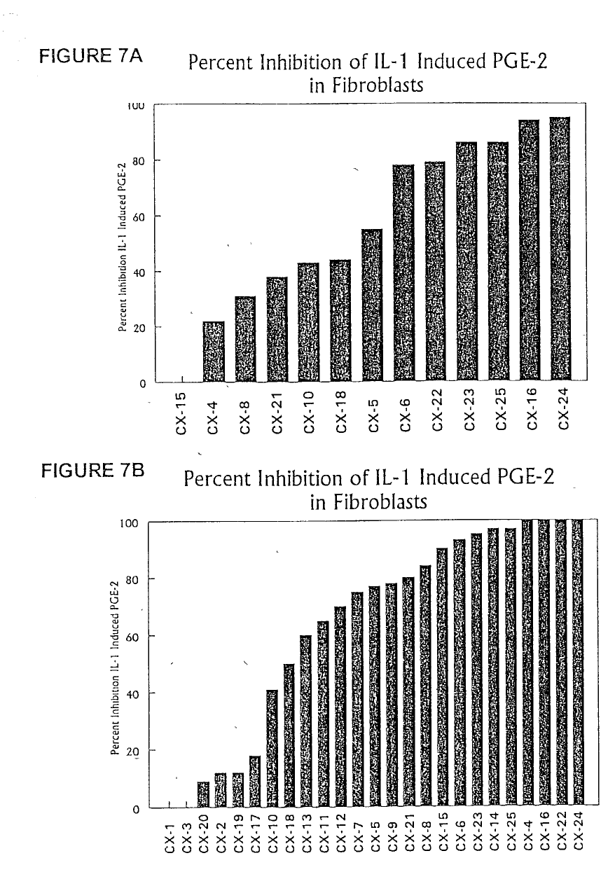

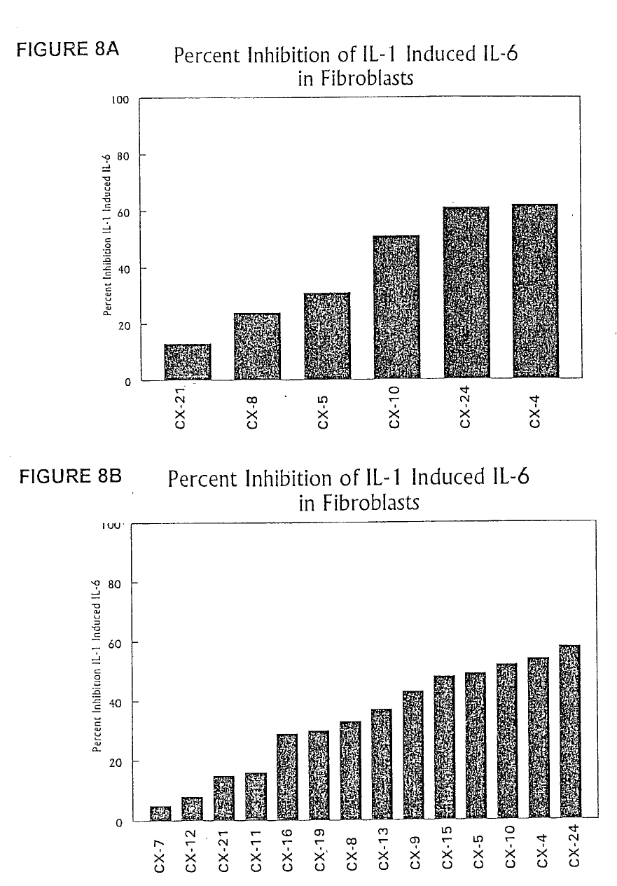

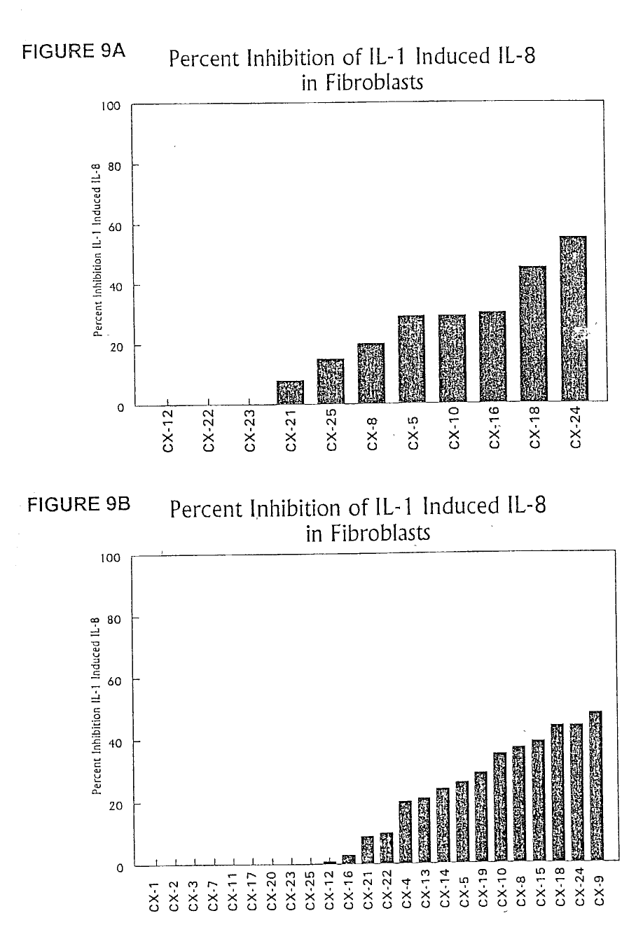

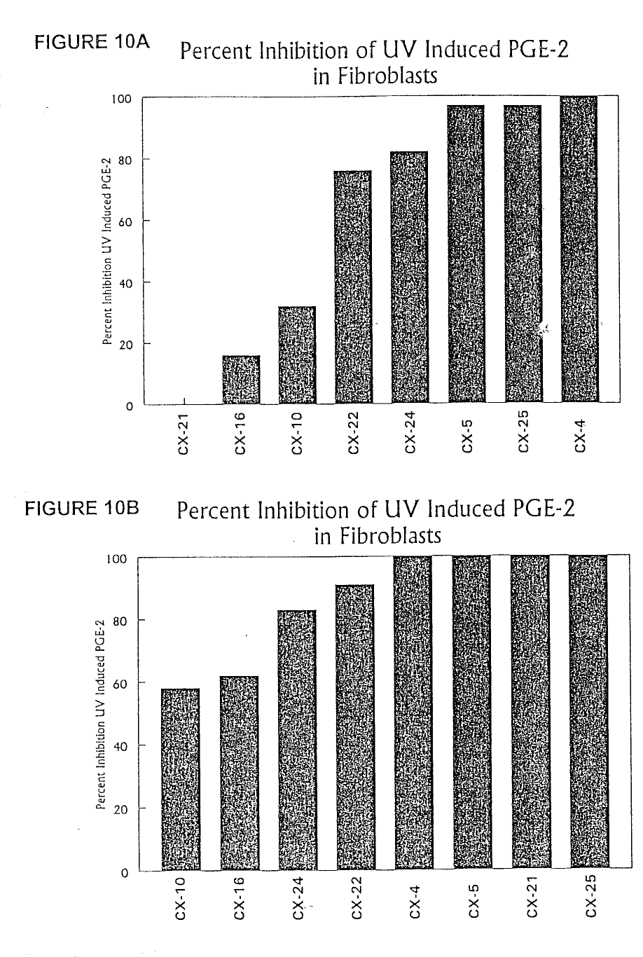

FIGS. 7A, 7B, 8A, 8B, 9A, 9B, 10A and 10B: Bar graphs of data tabulated in FIG. 5.

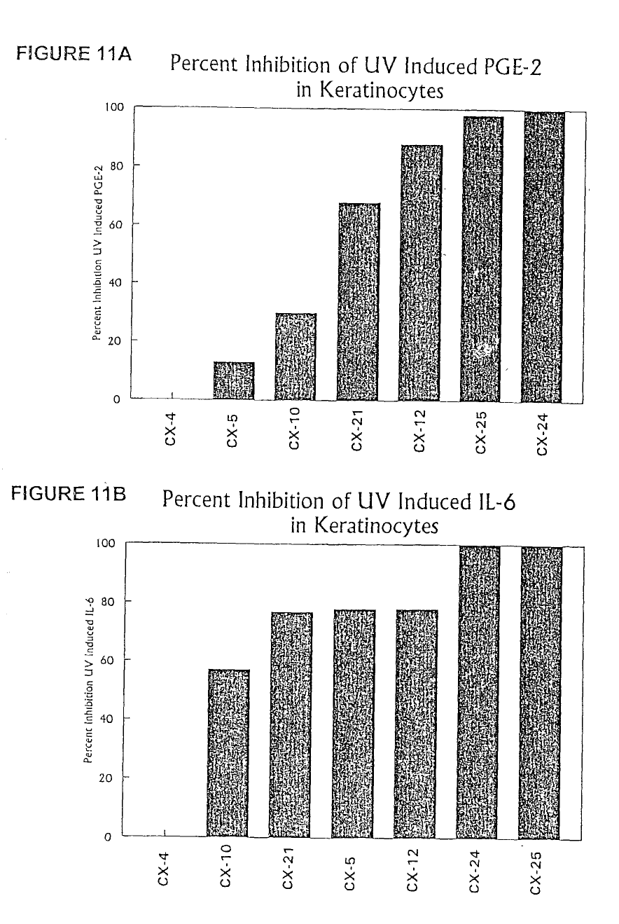

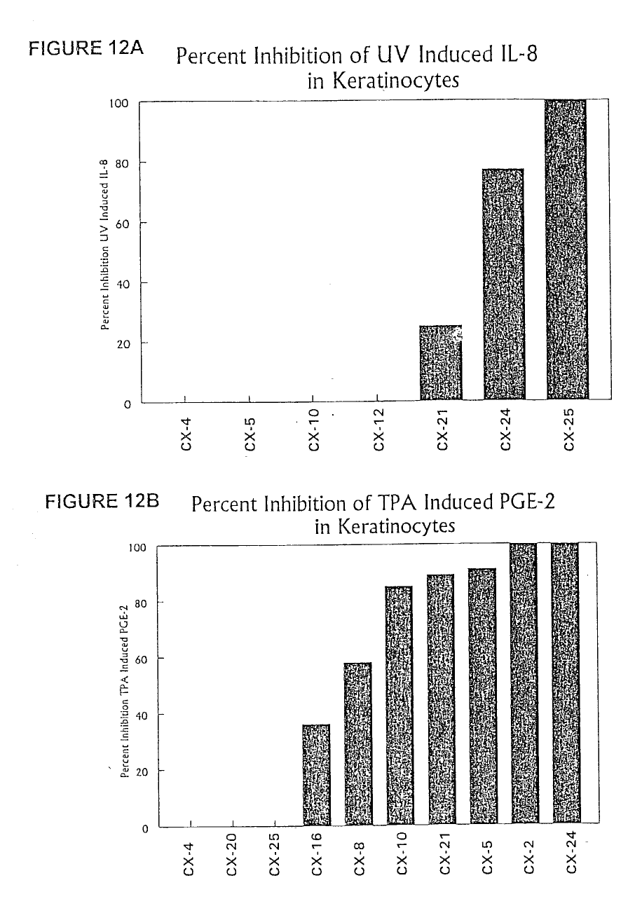

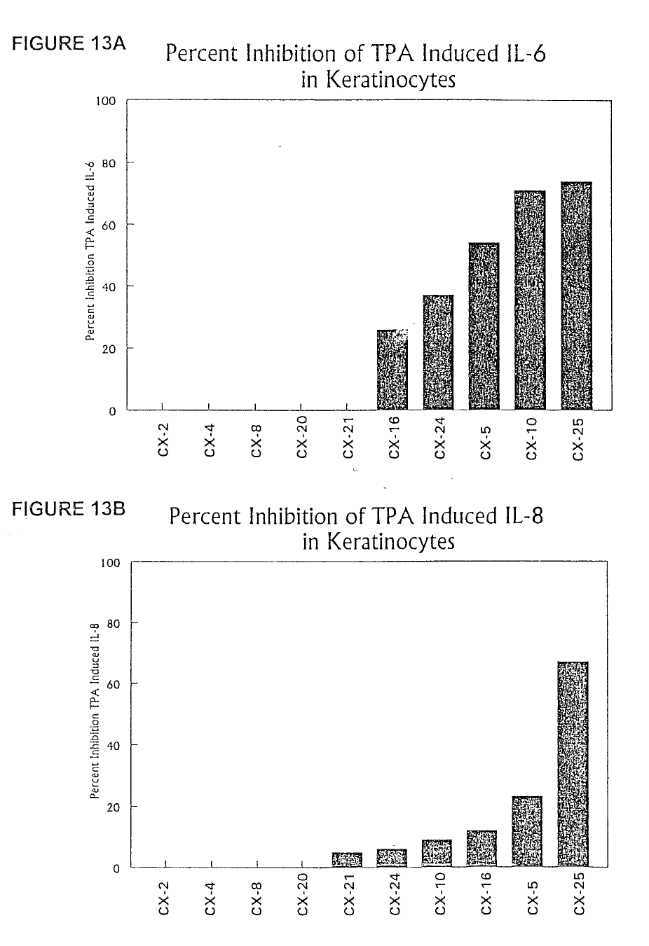

FIGS. 11A, 11B, 12A, 12B, 13A and 13B: Bar graphs of data tabulated in FIG. 6.

### DEFINITIONS

When describing the aromatic aldehyde compounds employed in the cosmetic and pharmaceutical compositions and methods of this invention as well as the compositions and methods themselves, the following terms have the following meanings:

“Aromatic aldehyde” refers to compounds that contain an aryl ring and an aldehyde group or an aldehyde group protected as an acetal or hemiacetal pendent from the ring.

“Acyl” refers to the group —C(O)R where R is hydrogen, alkyl or aryl. When R is hydrogen this is a “formyl”, when R is CH₃ this is “acetyl”.

“Alkyl” refers to monovalent saturated aliphatic hydrocarbon groups preferably having from 1 to about 20 carbon atoms, more preferably from 1 to 12, even more preferably 1 to 8 carbon atoms. This term is exemplified by groups such as methyl, ethyl, n-propyl, isopropyl, n-butyl, isobutyl, tert-butyl, n-hexyl, n-octyl, tert-octyl and the like. The term “lower alkyl” refers to alkyl groups having 1 to 6 carbon atoms and especially 1 to 4 carbon atoms.

“Substituted alkyl” refers to an alkyl group, preferably of from 1 to 10 carbon atoms, having from 1 to 5 substituents, and preferably 1 to 3 substituents, selected from the group consisting of alkoxy, cycloalkyl, cycloalkoxy, acyl, aminoacyl, amino, aminocarbonyl, cyano, halogen, hydroxyl, carboxyl, keto, thioketo, alkoxycarbonyl, thiol, thioalkoxy, aryl, aryloxy, nitro, —OSO₃H and pharmaceutically acceptable salts thereof, —SO-alkyl, —SO-substituted alkyl, —SO-aryl, —SO₂-alkyl, —SO₂-substituted alkyl, —SO₂-aryl, and mono- and di-alkylamino, mono- and di-arylamino, and unsymmetric di-substituted amines having different substituents selected from alkyl, substituted alkyl and aryl.

“Alkylene” refers to divalent alkylene groups preferably having from 1 to 10 carbon atoms and more preferably 1 to 6 carbon atoms which can be straight chain or branched. This term is exemplified by groups such as methylene (—CH₂—), ethylene (—CH₂CH₂—), the propylene isomers (e.g., —CH₂CH₂CH₂— and —CH(CH₃)CH₂—) and the like.

“Alkcycloalkyl” refers to -alkylene-cycloalkyl groups preferably having from 1 to 10 carbon atoms in the alkylene moiety and from 3 to 8 carbon atoms in the cycloalkyl moiety. Such alkcycloalkyl groups are exemplified by —CH₂-cyclopropyl, —CH₂-cyclopentyl, —CH₂CH₂-cyclohexyl, and the like.

“Alkcycloalkoxy” refers to —O-alkylene-cycloalkyl groups preferably having from 1 to 10 carbon atoms in the alkylene moiety and from 3 to 8 carbon atoms in the cycloalkyl moiety. Such alkcycloalkoxy groups are exemplified by —OCH₂-cyclopropyl, —OCH₂-cyclopentyl, —OCH₂CH₂-cyclohexyl, and the like.

“Alkoxy” refers to the group “alkyl-O—”. Preferred alkoxy groups include, by way of example, methoxy, ethoxy, n-propoxy, isopropoxy, n-butoxy, tert-butoxy, sec-butoxy, n-pentyloxy, n-hexyloxy, 1,2-dimethylbutoxy, and the like.

“Alkoxycarbonyl” refers to the group —C(O)OR where R is alkyl.

“Aminocarbonyl” refers to the group —C(O)NRR where each R is independently hydrogen or alkyl.

“Aminoacyl” refers to the group —NRC(O)R where each R is independently hydrogen or alkyl.

“Aryl” refers to an unsaturated aromatic carbocyclic group of from 6 to 14 carbon atoms having a single ring (e.g., phenyl) or multiple condensed rings (e.g., naphthyl or anthryl). Preferred aryls include phenyl, naphthyl and the like.

Unless otherwise constrained by the definition for the individual substituent, such aryl groups can optionally be substituted with from 1 to 3 substituents selected from the group consisting of alkyl, alkoxy, alkaryloxy, alkenyl, alkynyl, amino, aminoacyl, aminocarbonyl, alkoxycarbonyl, aryl, carboxyl, cycloalkoxy, cyano, halo, hydroxy, nitro, trihalomethyl, thioalkoxy, and the like.

“Aryloxy” refers to —O-aryl groups wherein “aryl” is as defined above.

“Carboxyl” refers to the group —C(O)OH.

“Cyano” refers to the group —CN.

“Cycloalkyl” refers to cyclic alkyl groups of from 3 to 10 carbon atoms having a single cyclic ring or multiple condensed rings, including fused and bridged ring systems, which can be optionally substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, or multiple ring structures such as adamantanyl, and the like.

“Cycloalkoxy” refers to —O-cycloalkyl groups. Such cycloalkoxy groups include, by way of example, cyclopentyloxy, cyclohexyloxy and the like.

“Heterocycloalkyl” refers to saturated cyclic groups of from 2 to 10 carbon atoms and 1, 2 or 3 heteroatoms selected from nitrogen, sulfur, or phosphorous, especially oxygen, for example. The ring can be optionally substituted with from 1 to 3 alkyl groups. Such heterocycloalkyl groups include, by way of example, single ring structures such as tetrahydrofuran, 1,4 dioxacyclopentanyl, dioxane, pyrrolidine, tetrahydrothiophene, and the like.

“Ionizing radiation” refers to any radiation that ionizes the atoms or molecules of matter. It may consist of particles (such as electrons) or it may be electromagnetic (ultraviolet radiation; X-rays; gamma radiation). Ionizing radiation occurs naturally, for example as a component of sunlight, and is emitted by radioactive substances. It is also produced artificially in X-ray machines, particle accelerators, nuclear reactors, etc.

“Isolated”, when used to define the state of purity of the aromatic aldehyde compounds used in the practice of this invention, means that the aromatic aldehyde has been substantially freed of (i.e at least about 90% and especially at least about 95% freed of) or separated from related feedstocks, co-products, or in the case of naturally-occurring mixtures, related materials with which the aldehyde appears in nature.

“Pharmaceutically-acceptable topical carrier” and equivalent terms refer to an inactive liquid or cream vehicle capable of suspending or dissolving the aromatic aldehyde and having the properties of being nontoxic and noninflammatory when applied to the skin. This term is specifically intended to encompass carrier materials approved for use in topical cosmetics. Representative carriers include water, oils, both vegetable and mineral, cream bases, lotion bases, ointment bases and the like. These bases include suspending agents, thickeners, penetration enhancers, and the like. Their formulation is well known to those in the art of cosmetics and topical pharmaceuticals. Additional information concerning carriers can be found in Part 8 of Remington's Pharmaceutical Sciences, 17th edition, 1985, Mack Publishing Company, Easton, Pa., which is incorporated herein by reference.

“Therapeutically effective dose” means a dose of a composition of this invention which, when applied topically to the skin of a patient afflicted with a dermatologic or other cosmetic or medical condition, or when administered by another route results in an observable improvement in the patient's condition.

“Topical”, when used to define a mode of administration, means that a material is administered by being applied to the skin.

“Topically effective” means that a material, when applied to the skin, produces a desired pharmacological result either locally at the place of application or systemically as a result of transdermal passage of an active ingredient in the material.

The Aromatic Aldehydes

The aromatic aldehydes include the compounds of Formula I as well as their acetal and hemiacetal equivalents shown in Formulas II and III. At this line the base aldehydes of Formula I are preferred.

Preferably, in the aromatic aldehyde compounds of Formula I, II and III above, R¹ is selected from the group consisting of a carbon-oxygen single bond, methylene and ethylene. More preferably, R¹ is a carbon-oxygen single bond.

Preferably, R² is selected from the group consisting of a carbon-oxygen single bond, methylene and ethylene. More preferably, R² is a carbon-oxygen single bond.

Preferably, each R³ is independently alkyl, or in the case of the acetals of Formula II, the two R³s together with the atoms to which they are attached form a heterocycloalkyl such as 1,4-dioxacyclopentanyl or a substituted 1,4-dioxacyclopentanyl.

The four R⁴s preferably include at least 2 hydrogens. More preferably, the remaining two R⁴s are each independently, hydrogen, hydroxyl, alkyl or alkoxy.

A preferred group of compounds of Formula I, II and III are those in which R¹ is a carbon-carbon single bond; R² is a carbon-oxygen single bond located in the 2, 3 or 4 position on the aromatic ring relative to the aldehyde functionality, and at least two R^{4′}s are each hydrogen.

An especially preferred group of compounds of Formula I, II and III are those in which R¹ is a carbon-carbon single bond; R² is a carbon-oxygen single bond located in the 4 position on the aromatic ring relative to the aldehyde functionality, and two of the R^{4′}s are each alkyl. Of these, the base aldehydes of Formula I are presently preferred.

In another of its composition aspects, this invention is directed to the use of each of the following representative individual compounds in pharmaceuticals and cosmetics:

2-hydroxybenzaldehyde

3-methyl-2-hydroxybenzaldehyde

3-ethyl-2-hydroxybenzaldehyde

3-n-propyl-2-hydroxybenzaldehyde

3-isopropyl-2-hydroxybenzaldehyde

3-n-butyl-2-hydroxybenzaldehyde

3-sec-butyl-2-hydroxybenzaldehyde

3-tert-butyl-2-hydroxybenzaldehyde

3-amyl-2-hydroxybenzaldehyde

4-methyl-2-hydroxybenzaldehyde

4-ethyl-2-hydroxybenzaldehyde

4-n-propyl-2-hydroxybenzaldehyde

4-isopropyl-2-hydroxybenzaldehyde

4-n-butyl-2-hydroxybenzaldehyde

4-sec-butyl-2-hydroxybenzaldehyde

4-tert-butyl-2-hydroxybenzaldehyde

4-amyl-2-hydroxybenzaldehyde

5-methyl-2-hydroxybenzaldehyde

5-ethyl-2-hydroxybenzaldehyde

5-n-propyl-2-hydroxybenzaldehyde

5-isopropyl-2-hydroxybenzaldehyde

5-n-butyl-2-hydroxybenzaldehyde

5-sec-butyl-2-hydroxybenzaldehyde

5-tert-butyl-2-hydroxybenzaldehyde

5-amyl-2-hydroxybenzaldehyde

6-methyl-2-hydroxybenzaldehyde

6-ethyl-2-hydroxybenzaldehyde

6-n-propyl-2-hydroxybenzaldehyde

6-isopropyl-2-hydroxybenzaldehyde

6-n-butyl-2-hydroxybenzaldehyde

6-sec-butyl-2-hydroxybenzaldehyde

6-tert-butyl-2-hydroxybenzaldehyde

6-amyl-2-hydroxybenzaldehyde

3,5 dinitro-2-hydroxybenzaldehyde

3,5 difluoro-2-hydroxybenzaldehyde

3,4 diisobutyl-2-hydroxybenzaldehyde

3,4 di-tert-butyl-2-hydroxybenzaldehyde

3,6 di-tert-butyl-2-hydroxybenzaldehyde

3-hydroxybenzaldehyde

2-methyl-3-hydroxybenzaldehyde

2-ethyl-3-hydroxybenzaldehyde

2-n-propyl-3-hydroxybenzaldehyde

2-isopropyl-3-hydroxybenzaldehyde

2-n-butyl-3-hydroxybenzaldehyde

2-sec-butyl-3-hydroxybenzaldehyde

2-tert-butyl-3-hydroxybenzaldehyde

2-amyl-3-hydroxybenzaldehyde

4-methyl-3-hydroxybenzaldehyde

4-ethyl-3-hydroxybenzaldehyde

4-n-propyl-3-hydroxybenzaldehyde

4-isopropyl-3-hydroxybenzaldehyde

4-n-butyl-3-hydroxybenzaldehyde

4-sec-butyl-3-hydroxybenzaldehyde

4-tert-butyl-3-hydroxybenzaldehyde

4-amyl-3-hydroxybenzaldehyde

5-methyl-3-hydroxybenzaldehyde

5-ethyl-3-hydroxybenzaldehyde

5-n-propyl-3-hydroxybenzaldehyde

5-isopropyl-3-hydroxybenzaldehyde

5-n-butyl-3-hydroxybenzaldehyde

5-sec-butyl-3-hydroxybenzaldehyde

5-tert-butyl-3-hydroxybenzaldehyde

5-amyl-3-hydroxybenzaldehyde

6-methyl-3-hydroxybenzaldehyde

6-ethyl-3-hydroxybenzaldehyde

6-n-propyl-3-hydroxybenzaldehyde

6-isopropyl-3-hydroxybenzaldehyde

6-n-butyl-3-hydroxybenzaldehyde

6-sec-butyl-3-hydroxybenzaldehyde

6-tert-butyl-3-hydroxybenzaldehyde

6-amyl-3-hydroxybenzaldehyde

2,4 difluoro-3-hydroxybenzaldehyde

2,4 dicyano-3-hydroxybenzaldehyde

2,4 di-tert-butyl-3-hydroxybenzaldehyde

2,4 diisopropyl-3-hydroxybenzaldehyde

2,5 di-tert-butyl-3-hydroxybenzaldehyde

4-hydroxybenzaldehyde

2-methyl-4-hydroxybenzaldehyde

2-ethyl-4-hydroxybenzaldehyde

2-n-propyl-4-hydroxybenzaldehyde

2-isopropyl-4-hydroxybenzaldehyde

2-n -butyl-4-hydroxybenzaldehyde

2-sec-butyl-4-hydroxybenzaldehyde

2-tert-butyl-4-hydroxybenzaldehyde

2-amyl-4-hydroxybenzaldehyde

3-methyl-4-hydroxybenzaldehyde

3-ethyl-4-hydroxybenzaldehyde

3-n-propyl-4-hydroxybenzaldehyde

3-isopropyl-4-hydroxybenzaldehyde

3-n-butyl-4-hydroxybenzaldehyde

3-sec-butyl-4-hydroxybenzaldehyde

3-tert-butyl-4-hydroxybenzaldehyde

3-amyl-4-hydroxybenzaldehyde

3,5 diisopropyl-4-hydroxybenzaldehyde

2,6-difluoro-4-hydroxybenzaldehyde

3,5-difluoro-4-hydroxybenzaldehyde

3,5 di-tert-butyl-4-hydroxybenzaldehyde

3-ethoxy-4-hydroxybenzaldehyde

4-hydroxy-3,5-dimethoxybenzaldehyde

2-hydroxy-3,5-dichlorobenzaldehyde

2,6-dihydroxybenzaldehyde 2,4-dihydroxy-6-methylbenzaldehyde

2,4,6-trihydroxybenzaldehyde

5-chloro-2-hydroxybenzaldehyde

2-hydroxy-5-bromobenzaldehyde

3-chloro-4-hydroxybenzaldehyde

2-hydroxy-3,5-diiodobenzaldehyde

3-bromo-4-hydroxy-5-methoxybenzaldehyde

2,4-dihydroxy-3-methylbenzaldehyde

2-hydroxy-3-methoxy-6-bromobenzaldehyde

2,4-dihydroxy-5-propylbenzaldehyde

2,4-dihydroxy-5-hexylbenzaldehyde

3-hydroxy-4-carboxybenzaldehyde

2-formyl-3,6-dihydroxy-4,5-dimethylbenzaldehyde

chloro-4-hydroxy-3-methoxybenzaldehyde

2,3,6-trihydroxybenzaldehyde

2,4-dihydroxy-5-acetylbenzaldehyde

2-formyl-3,6-dihydroxy-4,5-dipropylbenzaldehyde

2-formyl-3-methoxy-4,5-dimethyl-6-hydroxybenzaldehyde

2,3,5-trihydroxybenzaldehyde

2-hydroxy-6-(oxy-4-methylpentanoic acid)benzaldehyde

3-formyl-4,5-dihydroxybenzaldehyde

2-ethyl-6-hydroxybenzaldehyde

3-chloro-5-(3,7-dimethyl-2,6-octadienyl)-4,6-dihydroxy-2-methylbenzaldehyde

2-hydroxy-6-(8-pentadecenyl)benzaldehyde

2,4-dihydroxy-3-ethyl-6-(1-methylpentyl)benzaldehyde

3-chloro-5-(3,7-dimethyl-5-oxo-2,6-octadienyl)-4,6-dihydroxy-2-methylbenzaldehyde

2-pentanoic acid-3-formyl-4,5-dihydroxy benzaldehyde

2-propanoic acid-3-formyl-4,5-dihydroxy benzaldehyde

2,3,4-trihydroxy-5-methyl-6-hydroxymethylbenzaldehyde

2-hydroxy-4-methoxybenzaldehyde

2-hydroxy-5-carboxybenzaldehyde

3-carboxy-4-hydroxybenzaldehyde

2,3-dihydroxy-4-methoxybenzaldehyde

2-hydroxy-6-methoxybenzaldehyde

2, 5-dihydroxybenzaldehyde

2,3,4-trihydroxy-6-hydroxymethylbenzaldehyde

3,5-dimethyl-4-hydroxybenzaldehyde

3,4,5-trihydroxybenzaldehyde

2,3-dihydroxybenzaldehyde

2-hydroxy-5-acetylbenzaldehyde

2-hydroxy-5-carboxyethylbenzaldehyde

2-hydroxy-5-carboxypropylbenzaldehyde

2-hydroxy-5-carboxybutylbenzaldehyde

3-carboxy-4-hydroxybenzaldehyde

2-carboxymethyl-3-hydroxybenzaldehyde

2-carboxyethyl-3-hydroxybenzaldehyde

2-hydroxy-3-iodo-5-carboxymethylbenzaldehyde

2-formyl-3,4,5-trihydroxybenzaldehyde

benzaldehyde dimethyl acetal

benzaldehyde glyceryl acetal

benzaldehyde propylene glycol acetal

and the like.

Preferred aldehydes include: 3,5-dihydroxybenzaldehyde, 3,5-di-tert-butyl-4-hydroxybenzaldehyde, 3-ethoxy-4-hydroxybenzaldehyde and 4-hydroxy-3,5-dimethoxybenzaldehyde.

Benzaldehyde dimethyl acetal, benzaldehyde glyceryl acetal, benzaldehyde propylene glycol acetal, and cuminaldehyde are synthetic flavoring substances approved by the Food and Drug Administration (FDA) for use in food for humans. The details for their use are discussed in 21 C.F.R. § 172.515 (2000).

General Synthetic Procedures

The aromatic aldehydes employed in the compositions and methods of this invention may be available commercially or can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

The aromatic aldehydes of Formula I, II and III employed in the compositions and methods are either known compounds or compounds that can be prepared from known compounds by conventional procedures. For example, such compounds are readily prepared by acylation of the corresponding aryl compound with the appropriate acyl halide under Friedel-Crafts acylation reaction conditions. Additionally, the formyl compounds, i.e. those compounds where R⁴ is hydrogen, can be prepared by formulation of the corresponding aryl compound using, for example, a disubstituted formamide, such as N-methyl-N-phenylformamide, and phosphorous oxychloride (the Vilsmeier-Haack reaction), or using Zn(CN)₂ followed by water (the Gatterman reaction). Numerous other methods are known in the art for preparing such aryl carbonyl compounds. Such methods are described, for example, in I. T. Harrison and S. Harrison, Compendium of Organic Synthetic Methods, Wiley, New York, 1971, and references cited therein.

Certain aromatic aldehyde compounds of Formula I can also be prepared by alkylation of the corresponding aryl hydroxy compound (e.g., 4-hydroxybenzaldehyde and the like). This reaction is typically conducted by contacting the aryl hydroxy compound with a suitable base, such as an alkali or alkaline earth metal hydroxide, fluoride or carbonate, in a inert solvent, such as ethanol, DMF and the like, to deprotonate the hydroxyl group. This reaction is generally conducted at about 0° C. to about 50° C. for about 0.25 to 2 hours. The resulting intermediate is then reacted in situ with about 1.0 to about 2.0 equivalents of an alkyl halide, preferably an alkyl bromide or iodide, at a temperature of from about 25° C. to about 100° C. for about 0.25 to about 3 days.

Additionally, various aromatic aldehydes of Formula I can be prepared by reduction of the corresponding aryl nitriles. This reaction is typically conducted by contacting the aryl nitrile with about 1.0 to 1.5 equivalents of a hydride reducing agent, such as LiAlH(OEt)₃, in an inert solvent such as diethyl ether, at a temperature ranging from about −78° to about 25° C. for about 1 to 6 hours. Standard work-up conditions using aqueous acid then provides the corresponding aryl aldehyde.

The aromatic aldehydes of Formula II and III employed in the compositions and methods are either known compounds or compounds that can be prepared from known compounds by conventional procedures. The hemiacetals can be formed by either acid or base catalyzed reaction of the corresponding aldehyde with and alcohol. If a single equivalent of the alcohol is added to the carbonyl, the hemiacetal is formed. Addition of 2 equivalents of an alcohol to the carbonyl produces the acetal. Acetal formation is acid catalyzed and is typically conducted by adding 1 mol of aldehyde and a 0.1 mol of CaCl₂ to 1.9 mol of ethanol. The reaction mixture is held at room temperature for 1 to 2 days. Standard work up conditions provide the acetal protected aromatic aldehyde.

Pharmaceutical and Cosmetic Compositions

The aromatic aldehydes are administered in the form of a pharmaceutical or cosmetic composition. Such compositions can be prepared in a manner well known in the pharmaceutical and cosmetic arts and comprise at least one active compound.

Generally, the compositions of this invention are administered in a cosmetic amount or a therapeutically effective dose. The amount of the compound actually administered in a therapeutic setting may, typically be determined by a physician, in the light of the relevant circumstances, including the condition to be treated, the chosen route of administration, the actual compound administered, the age, weight, and response of the individual patient, the severity of the patient's symptoms, and the like. In cosmetic settings, the amount to be applied is selected to achieve a desired cosmetic effect.

The cosmetic compositions of this invention are to be administered topically. The pharmaceutical compositions of this invention are to be administered topically, transdermally or systemically such as orally or by injection.

In such compositions, the aromatic aldehyde compound is usually a minor component (from about 0.001 to about 20% by weight or preferably from about 0.01 to about 10% by weight) with the remainder being various vehicles or carriers and processing aids helpful for forming the desired dosing form.

Topical cosmetic forms and topical pharmaceutical dosing forms can include lotions, shampoos, soaks, gels, creams, ointments and pastes. Lotions commonly employ a water or alcohol base. Gels are semi-solid emulsions or suspensions. Creams generally contain a significant proportion of water in their base which ointments and creams are more commonly more oily.

Liquid forms, such as lotions suitable for topical administration or for cosmetic application, may include a suitable aqueous or non-aqueous vehicle with buffers, suspending and dispensing agents, thickeners, penetration enhancers, and the like. Solid forms such as creams or pastes or the like may include, for example, any of the following ingredients, water, oil, alcohol or grease as a substrate with surfactant, polymers such as polyethylene glycol, thickeners, solids and the like. Liquid or solid formulations may include enhanced delivery technologies such as liposomes, microsomes, microsponges and the like.

The above-described components for liquid, semisolid and solid topical compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

When pharmaceutical compositions are to be administered transdermally they typically are employed as liquid solutions or as gels. In these settings the concentration of active aldehyde ranges from about 0.1% to about 20%, and preferably from about 0.1% to about 5%, of the composition with the remainder being aqueous mixed or nonaqueous vehicle, such as alcohols and the like, suspending agents, gelling agents, surfactant, and the like. Examples of suitable such materials are described below.

The aldehyde-containing compositions of this invention can also be administered in sustained release transdermal forms or from transdermal sustained release drug delivery systems. A description of representative sustained release materials can be found in the incorporated materials in Remington's Pharmaceutical Sciences.

The compositions for systemic administration include compositions for oral administration, that is liquids and solids, and compositions for injection.

Compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical occupant. Typical unit dosage forms include profiled, premeasured ampules or syringes of the liquid compositions or pills, tablets, capsules or the like in the case of solid compositions. In such compositions, the aromatic aldehyde is usually a minor component (from about 0.01 to about 20% by weight or preferably from about 0.1 to about 15% by weight) with the remainder being various vehicles or carriers and processing aids helpful for forming the desired dosing form.

Liquid forms suitable for oral administration may include a suitable aqueous or non-aqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an occupant such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the aromatic aldehyde in such compositions is typically a minor component, often being from about 0.005 to 5% by weight with the remainder being the injectable carrier and the like.

The above-described components for orally administrable or injectable compositions are merely representative. Other materials as well as processing techniques and the like are set forth in the part of Remington's Pharmaceutical Sciences noted above.

The following formulation examples illustrate representative cosmetic and pharmaceutical compositions of this invention. The present invention, however, is not limited to the following pharmaceutical compositions.

### Formulation 1—Liquid

A compound of Formula I, II or III (125 mg), and xanthan gum (4 mg) are blended, passed through a No. 10 mesh U.S. sieve, and then mixed with a previously made solution of microcrystalline cellulose and sodium carboxymethyl cellulose (11:89, 50 mg) in a water/isopropanol (75:25) mixture. Sufficient water/isopropanol are then added to produce a total volume of 5 mL.

### Formulation 2—Cream

A commercial mineral oil-water cold cream base is obtained. To 100 grams of this base, 0.75 grams of a compound of Formula I, II or III as a fine powder or liquid, is added with continuous mixing and stirring to suspend the powder in the base and yield a pharmaceutical composition.

This composition includes the following: deionized water (57.6% by weight); niacinamide (2.0%); glycerin (4.0%); phenonip (1.0%); propylene glycol (5.0%); transcutol (3.2%); jojoba Oil (3.5%); isocetyl alcohol (2.0%); isocetyl stearate (3.5%); mineral oil (3.0%); 4-ethoxybenzaldehyde (1.0%); isostearyl palmitate (3.0%); PEG-7 glyceryl cocoate (2.0%); Glycereth-7 (2.0%); POLYSORBATE-20™ (0.2%); cetyl ricinoleate (1.0%); glyceryl stearate/PEG-100 stearate (4.0%); and SEPIGEL™ (2.0%).

### Formulation 3—Tablets

A compound of Formula I, II or III is mixed with dry gelatin binder and starch diluent in a 0.1: 1:1 weight ratio. A lubricating amount of magnesium stearate is added and the mixture is tabletted into 210 mg tablets containing 10 mg of active aromatic aldehyde.

### Formulation 4—Injection

A compound of Formula I, II or III is dissolved in injectable aqueous saline medium at a concentration of 1 mg/ml.

Utility and Dosing

The composition and methods of this invention can be used topically to treat dermatological conditions such as

actinic keratosis,

acne,

allergic contact dermatitis,

atopic eczema,

contact dermatitis,

eczema,

erythema,

hand eczema,

itch,

irritant contact dermatitis,

psoriasis,

seborrhoric eczema,

rosacea,

alopecia areata,

damage from radiation, including UV radiation, IR radiation and any other ionizing radiation

and the like.

The compositions, both cosmetic and pharmaceutical, can also be used to treat and prevent sunburn and to treat and prevent other forms of UV-induced inflammation and damage and damage from other forms of ionizing radiation.

In these applications the cosmetic and pharmaceutical compositions are administered topically to achieve a desired cosmetic effect or a topical therapeutic effect.

In these uses the dose levels or application levels can be expressed in terms of the amount of active aromatic aldehyde delivered to the skin. For example, 1 to about 5 doses or applications per day, each containing from about 0.001 g to about 1 gram of active aldehyde can be used.

Alternatively, dose levels can be expressed in terms of the volume of formulated composition administered. For example, 1 to about 5 doses or applications per day, each containing from about 1 to about 30 grams of composition containing from about 0.01% to about 10% by weight of active aldehyde and especially from 0.02% to about 8% by weight.

When used in sun care products, such as sun-care lotion, the concentration of aldehyde can be as set forth above and the product can be applied as needed based on the intensity and duration of sun exposure.

Additionally, since the aromatic aldehydes have been discovered to effectively inhibit the release of cytokines, such a IL-1α, such compounds are useful for treating diseases characterized by an overproduction or a dysregulated production of cytokines, particularly IL-1α. Elevated levels of IL-1 and other cytokines are associated with a wide variety of inflammatory conditions, including rheumatoid arthritis, septic shock, erythema nodosum leprosy, septicemia, adult respiratory distress syndrome (ARDS), inflammatory bowel disease (IBD), uveitis, damage from ionizing radiation and the like.

The relationships between these cytokines and related materials and the inflammatory processes are described in more detail below at “Biology and Testing”.

In the case of transdermal administration to treat such inflammatory conditions, one can administer a quantity of composition to a surface area of skin suitable to achieve an active aldehyde concentration in the systemic bloodstream of from about 0.5 to about 1000 micromolar and especially from about 1 to about 500 micromolar.

Injection dose levels for treating inflammatory conditions range from about 0.01 mg/kg/hour to at least 1 mg/kg/hour, all for from about 1 to about 120 hours and especially 24 to 96 hours. A preloading bolus of from about 0.01 mg/kg to about 1 mg/kg or more may also be administered to achieve adequate steady state levels.

With oral dosing, one to five and especially two to four and typically three oral doses per day are representative regimens. Using these dosing patterns, each dose provides from about 0.01 to about 10 mg/kg of the aromatic aldehyde, with preferred doses each providing from about 0.01 to about 5 mg/kg.

The aromatic aldehydes can be administered as the sole active agent or they can be administered in combination with other agents.

Biology and Testing

The examples include a number of in vitro studies to investigate the ability of these aldehydes to block various inflammatory processes in the skin. For these studies primary human keratinocytes and dermal fibroblast cell strains have been used as well as THP-1 monocytes and the Jurkat T-cell derived cell line. The in vitro experiments used to assess the anti-inflammatory activities of the aldehydes were selected on the basis of current knowledge about the skin inflammatory process. FIG. 1 depicts the events involved in cutaneous inflammation.

Inflammation in the skin is characterized by itching, pain, redness, swelling and, frequently, rough and flaky skin. These symptoms result from changes in blood flow to the site of inflammation, increased vascular permeability, the migration of cells from the circulation into the tissue, and the release of soluble mediators including cytokines, prostaglandins and chemokines. Skin inflammation can be triggered by: 1) infection caused by bacteria, parasites, fungi, or viruses, 2) injury resulting from physical trauma including burns, UV and ionizing radiation, 3) contact with chemical irritants, and 4) exposure to a foreign body such as an allergen which triggers an immune response.

Inflammation can be characterized as acute or chronic. Acute skin inflammation can result from exposure to UV radiation (UVR), ionizing radiation or contact with chemical irritants and allergens. In contrast, chronic inflammation results from a sustained immune cell mediated inflammatory response. Acute inflammatory responses are typically resolved within 1 to 2 weeks with little accompanying tissue destruction. Chronic inflammatory responses, however, are long-lasting because the antigen that triggered the response persists in the skin. This leads to continued recruitment of immune cells into the tissue, particularly T lymphocytes, which then produce and secrete high levels of many inflammatory mediators. Chronic inflammation leads to significant and serious tissue destruction.

Regardless of the stimulus that triggers either an acute or chronic cutaneous inflammatory response, the initial events are similar and are shown in FIGS. 1 and 2. Triggering stimuli, such as UV radiation, induce keratinocytes in the skin to produce various cytokines including the key inflammatory cytokine, Interleukin-1 (IL-1). These cells also produce Tumor Necrosis Factor (TNF-α) and prostaglandin E2 (PGE-2). PGE-2 causes vasodilation of blood vessels near the site of injury and also increases the sensitivity of sensory nerve endings resulting in the sensation of itching and pain. The principal action of TNF-α is to increase the production of adhesion molecules on the surface of endothelial cells lining the blood vessels. These adhesion molecules act as anchors within the blood vessel allowing immune cells moving through the circulation to attach to the endothelium, an event that can lead to the diapedsis (movement) of these cells from the circulation and into the tissue. IL-1 produced by keratinocytes binds to specific receptors on fibroblasts within the dermis and activates signaling pathways that lead to the induction of many pro-inflammatory genes, such as those for COX-2, IL-8 and IL-6. IL-1 also binds to specific receptors on mast cells resulting in the production and secretion of histamine (which also increases nerve ending sensitivity), cytokines and other inflammatory mediators. In addition to responding to keratinocyte-derived IL-1, fibroblasts can also be directly activated by the triggering stimulus (e.g. UVR) and this further stimulates the expression of pro-inflammatory genes resulting in the production of PGE-2, the chemokine IL-8, as well as collagenase-1 (MMP-1). IL-8 stimulates diapedsis (chemotaxis, movement) of neutrophils, monocytes and ultimately lymphocytes from the endothelial cells where they have attached as a result of the TNF-α induced increase in adhesion molecules. Once in the tissue, neutrophils and monocytes produce additional cytokines (IL-1, IL-12), and chemokines including monocyte chemotactic protein (MCP-1), a potent chemokine that accelerates the movement of monocytes into the tissue and helps transform them into macrophages. Mature macrophages in turn produce a variety of matrix metalloproteinases (MMPs) that degrade extracellular matrix proteins and thus reduce the strength, elasticity and thickness of the skin.

If the inflammatory response is maintained by the continued presence of an antigen in the skin as is the case with chronic and destructive cutaneous diseases such as psoriasis and atopic dermatitis, the persistence of the antigen causes T-lymphocytes to enter the tissue site and become activated. This activation leads to the production of cytokines such as TNF-α, monocyte chemotactic protein-1 (MCP-1), IL-8, IL-12, and interferon-γ (INF-γ). Released IL-12 causes the T-lymphocytes to proliferate rapidly and to produce a wide range of cytokines, growth factors and other inflammatory mediators. These released products further activate macrophages, recruit monocytes, increase tissue destruction and cause accelerated and uncontrolled growth of skin cells, particularly keratinocytes. The result is pronounced skin inflammation with redness, pain, itching and scaling of the skin as the keratinocytes move rapidly to the surface and “flake off”. Further, the rapid shedding of keratinocytes at the surface compromises the barrier function of the stratum corneum resulting in water loss and dry skin.

A common finding in inflammation is that cells in the skin respond to inflammatory stimuli by activating either one of two intracellular signaling pathways (or in some cases both pathways). These pathways are commonly referred to as the Stress Activated Kinase (SAK) pathway and the NF-kB pathway. The SAK pathway leads to the activation of the AP-1 transcription factor, which then binds to and activates several inflammatory genes including COX-2, IL-6 and MCP-1. Activation of the NF-kB pathway results in NF-kB protein translocation to the nucleus and activation of NF-KB driven inflammatory genes such as IL-8, MMP-1, TNF-α and the adhesion molecule, VCAM-1. Interestingly, many inflammatory genes including IL-1 have promoter elements that bind both AP-1 and NF-kB transcription factors and are thus regulated to some extent by both signaling pathways. The Cutanix screening assays are designed to determine which pathway is blocked by the compound under investigation, or if both pathways are effectively inhibited. A compound with the capacity to block the transcription of inflammatory genes regulated by each of these pathways will likely provide significant anti-inflammatory effects when applied topically. For each putative anti-inflammatory compound under consideration the initial screening program concentrates on the following target sites for intervention:

1. Inhibiting the production of IL-1 and PGE-2 in UVR or Tetradecanoyl Phorbol Acetate-treated keratinocytes.

2. Inhibiting the production of PGE-2 in UVR treated dermal fibroblasts.

3. Inhibiting the induction of PGE-2 in IL-1 treated fibroblasts.

Because one of the most common activators of skin inflammation is sunlight, specifically UVB radiation, the determination of a compound's ability to block the induction of pro-inflammatory PGE-2 by UVR in both keratinocytes and fibroblasts represents a logical first step in the screening process. In addition, because skin inflammation is often triggered by contact with chemical irritants or allergens, the use of TPA, which is known to trigger an inflammatory response in the skin, provides an additional model for the analysis of anti-inflammatory activities of test compounds. Finally, because IL-1 is one of the most important mediators and propagators of inflammation and is rapidly induced by an inflammatory stimulus, such as UVR, determining the ability of a potential anti-inflammatory compound to block either the production or action of IL-1 is a critically important initial screening study. As shown in FIGS. 1 and 2, by blocking IL-1 production from keratinocytes, not only is the activation of fibroblasts suppressed but the activation of mast cells is also blocked thus preventing the release of histamine and other inflammatory mediators. Furthermore, inhibition of IL-1 production in the skin would prevent the activation of a large number of inflammatory genes that are stimulated solely by IL-1. These include COX-2, MMP-1, and a variety of cytokine and chemokine genes.

For all of the initial screening studies described herein, cells in culture are exposed to the appropriate agonist, (i.e. UVR, TPA or IL-1) and then incubated in medium for 24 or 48 hours in the presence or absence of the compound under investigation. At 24 and 48-hour time points, medium from the cells is removed and assayed for a number of inflammatory mediators by ELISA.

Only primary keratinocyte and fibroblast cell strains were used, not immortalized cell lines, for the screening studies. The use of normal cells from the skin increases the probability that results from in vitro studies will be predictive of effects of a given compound when applied topically.

Aldehydes that are found to completely (100%) suppress PGE-2 induction at a concentration of 100 micromolar or less are then subjected to more demanding dose-response studies including the following sequence of experiments:

1. Assessment by ELISA of a compound's ability to block a variety of UVR, TPA, or IL-1 induced inflammatory mediators in keratinocytes and fibroblasts including IL-6, TNF-α, IL-8, and MMP-1.

2. Assessment by ELISA of a compound's ability to block the production and secretion of inflammatory mediators by monocytes (THP-1 monocyte line) stimulated by lipopolysaccharide (LPS) and by T lymphocytes (Jurkat cells) stimulated with an antibody ligand that activates the cells.

3. The use of RPA (ribonuclease protection analysis) to determine if a compound is acting at the gene level to suppress the activity of specific inflammatory genes stimulated by exposure of cells to various agonists including UVR, IL-1, TPA, or LPS (lipopolysaccharide). Cutanix has developed a customized RPA “cocktail” for keratinocytes, fibroblasts, T-cells, and monocytes to simultaneously measure the expression of cell-type specific inflammatory genes in cells stimulated with UVR, IL-1, TPA or LPS in the presence or absence of the compound under investigation.

4. The use of microarray gene analysis to simultaneously examine the effect of any compound on the expression of more than 5,500 genes specific for cells present in the skin. The gene arrays used were purchased from Research Genetics and provide read-outs on genes known to be expressed in the skin.

The aldehydes can suppress a number of pro-inflammatory mediators and FIG. 2 identifies some of the events that are likely inhibited by the aldehydes in vivo (shown by the circled X).

### EXAMPLES

The following examples are provided to further describe the invention and are not intended as limitations on the scope of the invention which is defined by the appended claims.

### EXAMPLE 1

In vitro experiments were conducted to demonstrate the activity of 3,5 di-tert-butyl, 4-hydroxybenzaldehyde, (“DTHB”) as a topically administered pharmaceuticals.

For this experiment, human skin fibroblasts were seeded into 12 well culture dishes at a density of 80,000 cells/well in tissue culture medium and left overnight to attach to the dish. The next day, medium was removed and replaced with fresh medium containing either 1% ethanol as a diluent control, IL-1 at a concentration of 500 picograms/ml, or IL-1 plus one of the compounds under investigation at a concentration of 1, 10, 50 or 100 μM. Cells were incubated for an additional 24 hours and at this time, the medium was removed and assayed by ELISA for the presence of PGE-2 in the culture medium. The results show that IL-1 caused a 4 to 22 fold increase in PGE-2.

The detailed results of studies comparing the activity of DTHB to 4-ethoxybenzaldehyde (“4-EB”), are shown in FIG. 3 wherein the percent inhibitions are as follows: 4-EB, 100%, 6% and 10% at 50 μM, 10 μM and 1 μM; DTHB 100%, 44.4% and 3.3% at 50 μM, 10 μM and 1 μM.

### EXAMPLE 2

Similar in vitro studies as those described in Example 1 were run using human skin keratinocytes except that the aromatic aldehyde was not added t. The experimental set up was the same as described for Example 1, but replacing IL-1 with tetradecanoyl phorbol acetate (TPA) at a concentration of 32 nM as the agonist. Samples of 3,5-Di-tert-butyl, 4- hydroxybenzaldehyde (DTHB) in concentrations of either 10, 50, or 100 μM were tested. The results show that TPA caused a 3.5 fold increase in PGE-2. However, treatment with DTHB blocked PGE-2 production by at least 50. The detailed results of studies comparing DTHB to 4-EB are shown in FIG. 4. The percent inhibitions are as follows: DTHB, 87.9% at 10 μM; 4-EB, 94.9% and 79.9% at 100 μM and 50 μM.

### EXAMPLE 3

In vitro experiments can be conducted to demonstrate the activity of the aromatic aldehyde DTHB as a topically administered pharmaceutical.

For this experiment, human skin fibroblasts should be seeded into 12 well culture dishes at a density of 80,000 cells/well in tissue culture medium and left overnight to attach to the dish. The next day, remove the medium and replace with fresh medium containing either 1% ethanol as a diluent control, IL-1 at a concentration of 500 picograms/ml, or IL-1 plus DTHB at either 250 μM or 500 μM. Incubate cells for an additional 24 hours then, remove the medium and assay by ELISA for the presence of PGE-2 in the culture medium.

### EXAMPLE 4

To determine the dose-response of human skin fibroblasts to DTHB, experiments as detailed above can be performed. The amount of DTHB that is added to the cells following the IL-1 dosing should be varied from about 250 μM to 1 μM.

### EXAMPLE 5

In vitro experiments were conducted to demonstrate the activity of a series of aromatic aldehydes as a topically administered pharmaceuticals. The compounds tested and the measured results are tabulated in FIG. 5, and shown graphically in FIGS. 7-10. These data include results from hydroxy compounds as used herein as well as other related compounds.

For this experiment, human skin fibroblasts were seeded into 12 well culture dishes at a density of 80,000 cells/well in tissue culture medium and left overnight to attach to the dish. The medium was then replaced with PBS for a challenge with either UV-light or with IL-1. After irradiation or introduction of IL-1, the PBS was removed and culture medium containing the appropriate compound (or DMSO for controls) was then added and the cells cultured for an additional 24 hours. At that time, the medium was removed and assayed by ELISA for the presence of PGE-2, IL-1, IL-6, IL-8, or MMP-1 in the culture medium. The levels of protein in the conditioned medium were measured and reported as percent inhibition relative to diluent controls.

IL-1 Challenge

On the second day, the medium was removed and replaced with fresh medium containing either 1% ethanol as a diluent control, IL-1 at a concentration of 500 picograms/ml, or IL-1 plus one of the compounds under investigation at a concentration of 100, 10, or 1 μM.

UV-light Challenge

On the second day, the medium was removed and replaced with fresh PBS for irradiation. The fibroblasts were then irradiated with 50 mJ of UVB. UVB irradiation was obtained by illuminating the samples with an FS-20 sunlamp through the lids of the multi-well plates in order to filter out the UVC radiation. After irradiation the PBS solution was removed and replaced with a solution containing either 1% ethanol as a diluent control, or one of the aldehyde compounds at a concentration of 100, 10, or 1 μM. The cells were incubated for another 24 hours and the medium was then removed for the ELISA assays and the cells were counted.

### EXAMPLE 6

Similar in vitro studies as those described in Example 5 were run using human skin keratinocytes. The experimental set up was the same as described for Example 5. The products assayed by ELISA for the presence of PGE-2, IL-1, IL-6, IL-8, MMP-1, or TNF-α in the culture medium.

For the cells challenged by a biochemical agonist, IL-1 was replaced with tetradecanoyl phorbol acetate (TPA) at a concentration of 32 nM. When UV-light was used to challenge the cells, they were exposed to 75 mj of UVB, obtained by illuminating the samples with an FS-20 sunlamp through the lids of the multi-well plates in order to filter out the UVC radiation.

The compounds tested were in concentrations of either 100, 10, or 1 μM, and the protein expression levels are reported in percent inhibition of growth.

The measured percent inhibitions are tabulated in FIG. 6 and shown graphically in FIGS. 11-13.

### EXAMPLE 7

In vivo studies can be carried out to determine if topically applied DTHB, or any other aromatic aldehyde of the present invention, could block an inflammatory response in humans. A topical lotion for any aromatic aldehyde of the present invention, may contain the following:

| Aqueous phase | | |
|---|---|---|
| Deionized water | 57.6% | (by weight) |
| Niacinamide | 2.0% | |
| Glycerin | 4.0% | |
| Phenonip | 1.0% | |
| Oil phase | | |
| Propylene glycol | 5.0% | |
| Transcutol | 3.2% | |
| Jojoba Oil | 3.5% | |
| Isocetyl alcohol | 2.0% | |
| Isocetyl Stearate | 3.5% | |
| Mineral Oil | 3.0% | |
| DTHB | 1.0% | |
| Isostearyl Palmitate | 3.0% | |
| PEG-7 Glyceryl Cocoate | 2.0% | |
| Glycereth-7 | 2.0% | |
| POLYSORBATE-20 ™ | 0.2% | |
| Cetyl Ricinoleate | 1.0% | |
| Glyceryl Stearate/ | 4.0% | |
| PEG-100 Stearate | | |
| Thickener | | |
| SEPIGEL ™ | 2.0% | |

This lotion should then be tested by Franz cell percutaneous absorption analysis to determine how much DTHB, or any other aromatic aldehyde of the present invention, can penetrate human skin over a 24 hour period.

This lotion should then be tested to determine if it could prevent an inflammatory response when applied topically to human skin. While the details are provided for a lotion containing DTHB, the same tests can be done for lotions containing any other aromatic aldehyde of the present invention.

For these studies a lab volunteer will be irradiated on a quarter sized spot on the inner forearm with 60-80 mJ of UVB light (a sunlamp). This dose is sufficient to cause a highly visible red erythema response. Immediately following irradiation on both arms, one arm is treated with the above DTHB lotion while the other arm is treated with the same lotion formulation, but with no DTHB. Within 2-6 hours after irradiation the vehicle-treated arm should develop a pronounced red erythema response at the site of irradiation while the DTHB lotion treated spot should not.

In addition to its anti-inflammatory activity compounds of the present invention, either alone or in combination with other compounds, such as ethyl vanillin, may have anti-aging properties. One of the classical symptoms of skin aging is an increase in collagenase activity in dermal fibroblasts which destroys collagen thereby leading to sagging skin and wrinkles.

Implications of the Results in terms of Potential Uses of the Discovery

Anti-aging

The finding that aromatic aldehydes of the present invention inhibit the activity of inflammatory genes in cultured skin cells and that they can block an inflammatory response in vivo when applied topically suggests wide utility for these compounds in the cosmetic, dermatology and oral drug markets. In the cosmetic market, these compounds when formulated for topical use can be expected to lower chronic sun-induced inflammation which causes the activation of genes in skin cells that destroy the skin matrix. By inhibiting sun-induced genes such as MMP-1 (collagenase), gelatinase, and cytokines IL-1, IL-12, etc. the compounds of the present invention will prevent the further breakdown of the skin and thus lessen the production of lines and wrinkles, sagging skin, and thinning of skin. It is likely that these aromatic aldehydes will stimulate genes that support the skin matrix such as collagen (studies ongoing). Thus, this product can be used as a “skin restorative” product for sun-damaged skin. It has its utility in treating actinic keratoses by both preventing their formation and actually reducing the size and number of existing keratoses.

Sun Care Products

Topically applied DTHB, or any other compound of this invention, may completely prevent the onset of a sunburn by UVB exposure, which suggests it may be used in sun care products including pre-sun, sun-tan lotions, and after-sun products. It is not suggested that the molecule has sun-screen properties (which it probably does to some extent) but that it can actually arrest the progression of a sunburn AFTER the skin has already been exposed to the UV rays of the sun. Although it has been shown that topical application of the product immediately after UVB exposure will prevent the onset of sunburn, it is also possible that application of the product even after the sunburn has appeared may: 1) prevent the continued progression of sunburn, and 2) reverse the redness already present.

What is claimed:

1. A composition comprising a pharmaceutically acceptable topical carrier and a compound of Formula I, II or III: 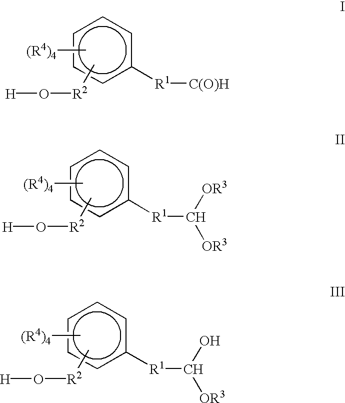
wherein
R¹ is a carbon-carbon single bond, or a straight chain or branched chain alkylene;
R² is a carbon-oxygen single bond, or a straight chain or branched chain alkylene;
each R³ is independently alkyl, or in the case of the compounds of Formula II, the two R³s together with the atoms to which they are attached form a heterocycloalkyl; and
each R⁴ is independently selected from the group consisting of hydrogen, hydroxyl, alkyl, substituted alkyl, alkcycloalkyl, cycloalkyl, alkoxy, alkcycloalkoxy, cycloalkoxy, acyl, acyloxy and halogen.

2. The composition according to claim 1 wherein R¹ is a carbon-carbon single bond.

3. The composition according to claim 1 wherein R¹ is a straight chain alkylene.

4. The composition according to claim 1 wherein R² is a carbon-oxygen single bond.

5. The composition according to claim 1 wherein R² is a straight chain alkylene.

6. The composition according to claim 2 wherein R² is a carbon-oxygen single bond.

7. The composition according to claim 2 wherein R² is a straight chain alkylene.

8. The composition according to claim 6 wherein at least 2 of the R⁴s are hydrogen.

9. The composition according to claim 8 wherein the compound of Formula I is: 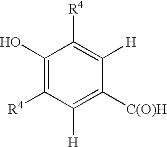
wherein each R⁴ is independently selected from the group consisting of hydrogen, alkyl, substituted alkyl, alkcycloalkyl, cycloalkyl, alkoxy, alkcycloalkoxy, cycloalkoxy.

10. The composition of claim 1 wherein the compound of Formula I, II or III is selected from the group consisting of
2-hydroxybenzaldehyde
3-methyl-2-hydroxybenzaldehyde
3-ethyl-2-hydroxybenzaldehyde
3-n-propyl-2-hydroxybenzaldehyde
3-isopropyl-2-hydroxybenzaldehyde
3-n-butyl-2-hydroxybenzaldehyde
3-sec-butyl-2-hydroxybenzaldehyde
3-tert-butyl-2-hydroxybenzaldehyde
3-amyl-2-hydroxybenzaldehyde
4-methyl-2-hydroxybenzaldehyde
4-ethyl-2-hydroxybenzaldehyde
4-n-propyl-2-hydroxybenzaldehyde
4-isopropyl-2-hydroxybenzaldehyde
4-n-butyl-2-hydroxybenzaldehyde
4-sec-butyl-2-hydroxybenzaldehyde
4-tert-butyl-2-hydroxybenzaldehyde
4-amyl-2-hydroxybenzaldehyde
5-methyl-2-hydroxybenzaldehyde
5-ethyl-2-hydroxybenzaldehyde
5-n-propyl-2-hydroxybenzaldehyde
5-isopropyl-2-hydroxybenzaldehyde
5-n-butyl-2-hydroxybenzaldehyde
5-sec-butyl-2-hydroxybenzaldehyde
5-tert-butyl-2-hydroxybenzaldehyde
5-amyl-2-hydroxybenzaldehyde
6-methyl-2-hydroxybenzaldehyde
6-ethyl-2-hydroxybenzaldehyde
6-n-propyl-2-hydroxybenzaldehyde
6-isopropyl-2-hydroxybenzaldehyde
6-n -butyl-2-hydroxybenzaldehyde
6-sec-butyl-2-hydroxybenzaldehyde
6-tert-butyl-2-hydroxybenzaldehyde
6-amyl-2-hydroxybenzaldehyde
3,5 dinitro-2-hydroxybenzaldehyde
3,5 difluoro-2-hydroxybenzaldehyde
3,4 diisobutyl-2-hydroxybenzaldehyde
3,4 di-tert-butyl-2-hydroxybenzaldehyde
3,6 di-tert-butyl-2-hydroxybenzaldehyde
3-hydroxybenzaldehyde
2-methyl-3-hydroxybenzaldehyde
2-ethyl-3-hydroxybenzaldehyde
2-n-propyl-3-hydroxybenzaldehyde
2-isopropyl-3-hydroxybenzaldehyde
2-n-butyl-3-hydroxybenzaldehyde
2-sec-butyl-3-hydroxybenzaldehyde
2-tert-butyl-3-hydroxybenzaldehyde
2-amyl-3-hydroxybenzaldehyde
4-methyl-3-hydroxybenzaldehyde
4-ethyl-3-hydroxybenzaldehyde
4-n-propyl-3-hydroxybenzaldehyde
4-isopropyl-3-hydroxybenzaldehyde
4-n-butyl-3-hydroxybenzaldehyde
4-sec-butyl-3-hydroxybenzaldehyde
4-tert-butyl-3-hydroxybenzaldehyde
4-amyl-3-hydroxybenzaldehyde
5-methyl-3-hydroxybenzaldehyde
5-ethyl-3-hydroxybenzaldehyde
5-n-propyl-3-hydroxybenzaldehyde
5-isopropyl-3-hydroxybenzaldehyde
5-n-butyl-3-hydroxybenzaldehyde
5-sec-butyl-3-hydroxybenzaldehyde
5-tert-butyl-3-hydroxybenzaldehyde
5-amyl-3-hydroxybenzaldehyde
6-methyl-3-hydroxybenzaldehyde
6-ethyl-3-hydroxybenzaldehyde
6-n-propyl-3-hydroxybenzaldehyde
6-isopropyl-3-hydroxybenzaldehyde
6-n-butyl-3-hydroxybenzaldehyde
6-sec-butyl-3-hydroxybenzaldehyde
6-tert-butyl-3-hydroxybenzaldehyde
6-amyl-3-hydroxybenzaldehyde
2,4 difluoro-3-hydroxybenzaldehyde
2,4 dicyano-3-hydroxybenzaldehyde
2,4 di-tert-butyl-3-hydroxybenzaldehyde
2,4 diisopropyl-3-hydroxybenzaldehyde
2,5 di-tert-butyl-3-hydroxybenzaldehyde
4-hydroxybenzaldehyde
2-methyl-4-hydroxybenzaldehyde
2-ethyl-4-hydroxybenzaldehyde
2-n-propyl-4-hydroxybenzaldehyde
2-isopropyl-4-hydroxybenzaldehyde
2-n-butyl-4-hydroxybenzaldehyde
2-sec-butyl-4-hydroxybenzaldehyde
2-tert-butyl-4-hydroxybenzaldehyde
2-amyl-4-hydroxybenzaldehyde
3-methyl-4-hydroxybenzaldehyde
3-ethyl-4-hydroxybenzaldehyde
3-n-propyl-4-hydroxybenzaldehyde
3-isopropyl-4-hydroxybenzaldehyde
3-n-butyl-4-hydroxybenzaldehyde
3-sec-butyl-4-hydroxybenzaldehyde
3-tert-butyl-4-hydroxybenzaldehyde
3-amyl-4-hydroxybenzaldehyde
3,5diisopropyl-4-hydroxybenzaldehyde
2,6-difluoro-4-hydroxybenzaldehyde
3, 5-difluoro-4-hydroxybenzaldehyde
3,5-dihydroxybenzaldehyde
2-hydroxy-3,5-dichlorobenzaldehyde
2,6-dihydroxybenzaldehyde
2,4-dihydroxy-6-methylbenzaldehyde
2,4,6-trihydroxybenzaldehyde
5-chloro-2-hydroxybenzaldehyde
2-hydroxy-5-bromobenzaldehyde
3-chloro-4-hydroxybenzaldehyde
2-hydroxy-3,5-diiodobenzaldehyde
3-bromo-4-hydroxy-5-methoxybenzaldehyde
2,4-dihydroxy-3-methylbenzaldehyde
2-hydroxy-3-methoxy-6-bromobenzaldehyde
2,4-dihydroxy-5-propylbenzaldehyde
2,4-dihydroxy-5-hexylbenzaldehyde
3-hydroxy-4-carboxybenzaldehyde
2-formyl-3,6-dihydroxy-4,5-dimethylbenzaldehyde
chloro-4-hydroxy-3-methoxybenzaldehyde
2,3,6-trihydroxybenzaldehyde
2,4-dihydroxy-5-acetylbenzaldehyde
2-formyl-3,6-dihydroxy-4,5-dipropylbenzaldehyde
2-formyl-3-methoxy-4,5-dimethyl-6-hydroxybenzaldehyde
2,3,5-trihydroxybenzaldehyde
2-hydroxy-6-(oxy-4-methylpentanoic acid)benzaldehyde
3-formyl-4,5-dihydroxybenzaldehyde
2-ethyl-6-hydroxybenzaldehyde
3-chloro-5-(3,7-dimethyl-2,6-octadienyl)-4, 6-dihydroxy-2-methylbenzaldehyde
2-hydroxy-6-(8-pentadecenyl)benzaldehyde
2-4-dihydroxy-3-ethyl-6-(1-methylpentyl)benzaldehyde
3-chloro-5-(3,7-dimethyl-5-oxo-2,6-octadienyl)-4,6-dihydroxy-2-methylbenzaldehyde
2-pentanoic acid-3-formyl-4,5-dihydroxy benzaldehyde
2-propanoic acid-3-formyl-4,5-dihydroxy benzaldehyde
2,3,4-trihydroxy-5-methyl-6-hydroxymethylbenzaldehyde
2-hydroxy-4-methoxybenzaldehyde
2-hydroxy-5-carboxybenzaldehyde
3-carboxy-4-hydroxybenzaldehyde
2,3-dihydroxy-4-methoxybenzaldehyde
2-hydroxy-6-methoxybenzaldehyde
2,5-dihydroxybenzaldehyde
2,3,4-trihydroxy-6-hydroxymethylbenzaldehyde
3,5-dimethyl-4-hydroxybenzaldehyde
3,4,5-trihydroxybenzaldehyde
2,3-dihydroxybenzaldehyde
2-hydroxy-5-acetylbenzaldehyde
2-hydroxy-5-carboxyethylbenzaldehyde
2-hydroxy-5-carboxypropylbenzaldehyde
2-hydroxy-5-carboxybutylbenzaldehyde
3-carboxy-4-hydroxybenzaldehyde
2-carboxymethyl-3-hydroxybenzaldehyde
2-carboxyethyl-3-hydroxybenzaldehyde
2-hydroxy-3-iodo-5-carboxymethylbenzaldehyde
2-formyl-3,4,5-trihydroxybenzaldehyde
benzaldehyde dimethyl acetal
benzaldehyde glyceryl acetal, and
benzaldehyde propylene glycol acetal.

11. The composition of claim 1 wherein the compound of Formula I is selected from the group consisting of 3,5-dihydroxybenzaldehyde, 3,5-di-tert-butyl-4-hydroxybenzaldehyde, 3-ethoxy-4-hydroxybenzaldehyde and 4-hydroxy-3,5-dimethoxybenzaldehyde.

12. The composition of claim 1 wherein the compound of Formula I is 3,5-dihydroxybenzaldehyde.

13. The composition of claim 1 wherein the compound of Formula I is 3,5-di-tert-butyl-4-hydroxybenzaldehyde.

14. The composition of claim 1 wherein the compound of Formula I is 3-ethoxy-4-hydroxybenzaldehyde.

15. The composition of claim 1 wherein the compound of Formula I is 4-hydroxy-3,5-dimethoxybenzaldehyde.

16. The composition of claim 1 wherein the composition is a cosmetic composition.

17. The cosmetic composition of claim 16 wherein the carrier is a liquid carrier.

18. The cosmetic composition of claim 16 wherein the carrier is a cream carrier.

19. The composition of claim 1 wherein the composition is a pharmaceutical composition and the compound is present in a pharmaceutically acceptable amount.

20. The pharmaceutical composition of claim 19 wherein the carrier is a liquid or a cream carrier.

21. The pharmaceutical composition of claim 19 wherein the pharmaceutical composition is a transdermal pharmaceutical composition and the compound is present in a transdermally effective amount.

22. The transdermal pharmaceutical composition of claim 21 wherein the carrier is a liquid carrier.

23. The transdermal pharmaceutical composition of claim 21 wherein the carrier is a cream carrier.

24. The transdermal pharmaceutical composition of claim 21 in a sustained release dosage form.

25. A systemic pharmaceutical composition comprising a systemically suitable pharmaceutically acceptable carrier and a pharmaceutically effective amount of a compound of Formula I, II or III: 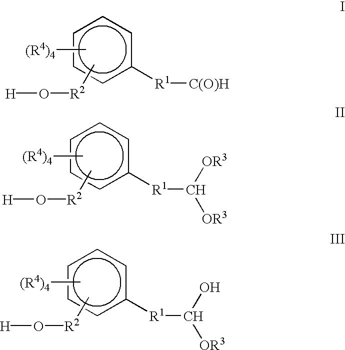
wherein
R¹ is a carbon-carbon single bond or a straight chain or branched chain alkylene;
R² is a carbon-oxygen single bond, or a straight chain or branched chain alkylene;
each R³ is independently alkyl, or in the case of the compounds of Formula II, the two R³s together with the atoms to which they are attached form a heterocycloalkyl; and
each R⁴ is independently selected from the group consisting of hydrogen, alkyl, substituted alkyl, alkcycloalkyl, cycloalkyl, alkoxy, alkcycloalkoxy, cycloalkoxy, acyl, acyloxy and halogen.

26. The composition of claim 25 wherein the carrier is an injectable carrier.

27. The composition of claim 25 wherein the carrier is a an oral liquid carrier.

28. The composition of claim 25 wherein the carrier is an oral solid carrier.

29. The composition of claim 28 in a unit dosage form.

30. The composition of claim 29 wherein the unit dosage form is a capsule.

31. The composition of claim 29 wherein the unit dosage form is a pill.

32. A method for treating a dermatologic condition which method comprises topically applying to a human an effective amount of the cosmetic composition of claim 16.

33. The method of claim 32 wherein the condition is an inflammatory condition.

34. A method for treating a patient with a dermatologic disease which method comprises topically administering to said patient a therapeutically effective amount of the topical pharmaceutical composition of claim 1.

35. A method of claim 34 wherein the disease is an inflammatory disease.

36. A method for treating a patient with an inflammatory disease which method comprises transdermally administering to said patient a therapeutically effective amount of the transdermal pharmaceutical composition of claim 21.

37. A method for treating a patient with an inflammatory disease which method comprises administering by injection to said patient a therapeutically effective amount of the injectable pharmaceutical composition of claim 26.

38. A method for treating a patient with an inflammatory disease which method comprises orally administering to said patient a therapeutically effective amount of an oral liquid pharmaceutical composition of claim 27.

39. A method for treating a patient with an inflammatory disease which method comprises orally administering to said patient a therapeutically effective amount of the oral solid pharmaceutical composition of claim 28.

40. A method for improving the skin appearance of a human which method comprises topically applying to human an effective skin appearance improving amount of the cosmetic composition of claim 16.

41. A method for improving the skin appearance of a patient which method comprises topically administering to a patient an effective skin appearance improving amount of the topical pharmaceutical composition of claim 1.
